(19) United States
(12) Patent Application Publication
(10) Pub. No.: US 2018/0362737 A1
(43) Pub. Date: Dec. 20, 2018

(54) COMPOSITIONS COMPRISING CELLULOSE ETHERS AND WATER-SOLUBLE ESTERIFIED CELLULOSE ETHERS

(71) Applicant: DOW GLOBAL TECHNOLOGIES LLC, Midland, MI (US)

(72) Inventors: Oliver PETERMANN, Hamburg (DE); Robert L. SCHMITT, Collegeville, PA (US)

(73) Assignee: Dow Global Technologies LLC, Midland, MI (US)

(21) Appl. No.: 16/060,158

(22) PCT Filed: Nov. 15, 2016
(86) PCT No.: PCT/US2016/061991

§ 371 Date: Jun. 7, 2018

Related U.S. Application Data
(60) Provisional application No. 62/264,411, filed on Dec. 8, 2015.

(51) Int. Cl. C08L 1/28; A61K 9/28; A61K 9/48

(52) U.S. Cl. CPC C08L 1/286; C08L 1/284; C08L 2201/54; A61K 9/4816; A61K 9/2866

(57) ABSTRACT

A composition comprising a) an esterified cellulose ether comprising aliphatic monovalent acyl groups and groups of the formula —C(O)—R—COOH, R being a divalent hydrocarbon group, wherein I) the degree of neutralization of the groups —C(O)—R—COOH is not more than 0.4 and II) the total degree of ester substitution is from 0.03 to 0.70, and b) a cellulose ether having a viscosity of from 1.2 to 200 mPa s, measured as a 2 weight-% aqueous solution at 20° C., gels at increased temperature and displays reduced syneresis when further increasing the temperature of the gel.

### FIELD

This invention concerns novel compositions comprising water-soluble esterified cellulose ethers and a method of reducing or preventing syneresis induced by temperature change of a gel formed from an aqueous solution of an esterified cellulose ether.

### INTRODUCTION

Esters of cellulose ethers, their uses and processes for preparing them are generally known in the art. When the esterified cellulose ethers comprise ester groups which carry carboxylic groups, the solubility of the esterified cellulose ethers in aqueous liquids is typically dependent on the pH. For example, the solubility of hydroxypropyl methyl cellulose acetate succinate (HPMCAS) in aqueous liquids is pH-dependent due to the presence of succinate groups, also called succinyl groups or succinoyl groups. HPMCAS is known as enteric polymer for pharmaceutical dosage forms. In the acidic environment of the stomach HPMCAS is protonated and therefore insoluble. HPMCAS undergoes deprotonation and becomes soluble in the small intestine, which is an environment of higher pH. Dosage forms coated with HPMCAS protect the drug from inactivation or degradation in the acidic environment of the stomach or prevent irritation of the stomach by the drug but release the drug in the small intestine. The pH-dependent solubility is dependent on the degree of substitution of acidic functional groups. The dissolution time of various types of HPMCAS dependent on pH and on the degree of neutralization of HPMCAS is discussed in detail in McGinity, James W. Aqueous Polymeric Coatings for Pharmaceutical Dosage Forms, New York: M. Dekker, 1989, pages 105-113. This publication illustrates in FIG. 16 on p. 112 the dissolution time of several grades of HPMCAS, which have different degrees of substitution with succinoyl, acetyl and methoxyl groups, in pure water and in 0.1 NaCl depending on the degree of neutralization of the HPMCAS. Depending on the HPMCAS and the presence or absence of NaCl, HPMCAS is soluble when it has a degree of neutralization between about 0.55 and 1. Below a degree of neutralization of about 0.55, all HPMCAS grades are insoluble in pure water and in 0.1 NaCl.

Co-pending International Patent Application WO 2016/148977, filed Mar. 8, 2016, claiming the priority of U.S. Provisional Application 62/133,514, filed Mar. 16, 2015 and International Patent Application WO 2016/148976, filed Mar. 8, 2016, claiming the priority of U.S. Provisional Application 62/133,518, filed Mar. 16, 2015, disclose novel esterified cellulose ethers which are soluble in water although the degree of neutralization of the carboxylic groups is not more than 0.4.—Aqueous solutions of many of these esterified cellulose ethers gel at slightly elevated temperature, typically at 30 to 55° C. This makes them very suitable for coating pharmaceutical dosage forms or for producing capsule shells. However, inventors of these patent applications have found that gels formed from aqueous solutions of such esterified cellulose ethers display expulsion of water from the gels at further increased temperatures, for example above 60° C., or more typically at 70° C. or more. This phenomenon is known as “syneresis”. In applications where gel formation is desired at elevated temperature, such as the production of capsules shells wherein heated dipping pins are used, syneresis is undesired as it causes a breakdown of the gel structure.

Therefore, there is a need to find a method of reducing or preventing syneresis induced by temperature change of a gel formed from an aqueous solution of an above-mentioned esterified cellulose ether.

### SUMMARY

One aspect of the present invention is a composition which comprises

a) an esterified cellulose ether comprising aliphatic monovalent acyl groups and groups of the formula —C(O)—R—COOH, R being a divalent hydrocarbon group, wherein I) the degree of neutralization of the groups —C(O)—R—COOH is not more than 0.4 and II) the total degree of ester substitution is from 0.03 to 0.70, and

b) a cellulose ether having a viscosity of from 1.2 to 200 mPa·s, measured as a 2 weight-% aqueous solution at 20° C. according to Ubbelohde.

Surprising, a gel formed from an aqueous solution comprising the above-mentioned esterified cellulose ether a) displays reduced or even no syneresis induced by temperature change of the gel when the gel is formed from an aqueous solution that comprises the above-mentioned cellulose ether b) in addition to the above-mentioned esterified cellulose ether a). Even more surprisingly, it has been found that the incorporation of the above-mentioned cellulose ether b) into the aqueous solution comprising the above-mentioned esterified cellulose ether a) does not reduce the storage modulus or gel strength of a gel formed from such aqueous solution to an undue degree.

Accordingly, another aspect of the present invention is method of reducing or preventing syneresis induced by temperature change of a gel formed from an aqueous solution of an esterified cellulose ether comprising aliphatic monovalent acyl groups and groups of the formula —C(O)—R—COOH, R being a divalent hydrocarbon group, wherein I) the degree of neutralization of the groups —C(O)—R—COOH is not more than 0.4, II) the total degree of ester substitution is from 0.03 to 0.70, wherein a cellulose ether having a viscosity of from 1.2 to 200 mPa·s, measured as a 2 weight-% aqueous solution at 20° C. according to Ubbelohde, is added to the aqueous solution before the gel is formed.

### BRIEF DESCRIPTION OF DRAWINGS

FIG. 1 illustrates the storage modulus of four aqueous compositions of the present invention and of two aqueous comparative compositions as a function of temperature.

FIG. 2 illustrates the storage modulus of five other aqueous compositions of the present invention and of two other aqueous comparative compositions as a function of temperature.

FIG. 3 illustrates the storage modulus of four other aqueous compositions of the present invention and of two other aqueous comparative compositions as a function of temperature.

FIG. 4 illustrates the storage modulus of five other aqueous compositions of the present invention and of two other aqueous comparative compositions as a function of temperature.

### DESCRIPTION OF EMBODIMENTS

Esterified cellulose ethers a) are described in copending International Patent Application International Patent Application WO 2016/148977, filed Mar. 8, 2016, which claims the priority of U.S. Provisional Application 62/133,514, filed Mar. 16, 2015 and International Patent Application WO 2016/148976, filed Mar. 8, 2016 which claims the priority of U.S. Provisional Application No. 62/133,518, filed on 16 Mar. 2015, all filed by the Applicants of the present patent application.

The esterified cellulose ether a) comprised in the composition of the present invention has a cellulose backbone having β-1,4 glycosidically bound D-glucopyranose repeating units, designated as anhydroglucose units in the context of this invention. The esterified cellulose ether a) preferably is an esterified alkyl cellulose, hydroxyalkyl cellulose or hydroxyalkyl alkylcellulose. This means that in the esterified cellulose ether a) comprised in the composition of the present invention, at least a part of the hydroxyl groups of the anhydroglucose units are substituted by alkoxyl groups or hydroxyalkoxyl groups or a combination of alkoxyl and hydroxyalkoxyl groups. The hydroxyalkoxyl groups are typically hydroxymethoxyl, hydroxyethoxyl and/or hydroxypropoxyl groups. Hydroxyethoxyl and/or hydroxypropoxyl groups are preferred. Typically one or two kinds of hydroxyalkoxyl groups are present in the esterified cellulose ether a). Preferably a single kind of hydroxyalkoxyl group, more preferably hydroxypropoxyl, is present. The alkoxyl groups are typically methoxyl, ethoxyl and/or propoxyl groups. Methoxyl groups are preferred. Illustrative of the above-defined esterified cellulose ether a) are esterified alkylcelluloses, such as esterified methylcelluloses, ethylcelluloses, and propylcelluloses; esterified hydroxyalkylcelluloses, such as esterified hydroxyethylcelluloses, hydroxypropylcelluloses, and hydroxybutylcelluloses; and esterified hydroxyalkyl alkylcelluloses, such as esterified hydroxyethyl methylcelluloses, hydroxymethyl ethylcelluloses, ethyl hydroxyethylcelluloses, hydroxypropyl methylcelluloses, hydroxypropyl ethylcelluloses, hydroxybutyl methylcelluloses, and hydroxybutyl ethylcelluloses; and those having two or more hydroxyalkyl groups, such as esterified hydroxyethylhydroxypropyl methylcelluloses. Most preferably, the esterified cellulose ether a) is an esterified hydroxyalkyl methylcellulose, such as an esterified hydroxypropyl methylcellulose.

The degree of the substitution of hydroxyl groups of the anhydroglucose units by hydroxyalkoxyl groups is expressed by the molar substitution of hydroxyalkoxyl groups, the MS(hydroxyalkoxyl). The MS(hydroxyalkoxyl) is the average number of moles of hydroxyalkoxyl groups per anhydroglucose unit in the esterified cellulose ether. It is to be understood that during the hydroxyalkylation reaction the hydroxyl group of a hydroxyalkoxyl group bound to the cellulose backbone can be further etherified by an alkylating agent, e.g. a methylating agent, and/or a hydroxyalkylating agent. Multiple subsequent hydroxyalkylation etherification reactions with respect to the same carbon atom position of an anhydroglucose unit yields a side chain, wherein multiple hydroxyalkoxyl groups are covalently bound to each other by ether bonds, each side chain as a whole forming a hydroxyalkoxyl substituent to the cellulose backbone.

The term “hydroxyalkoxyl groups” thus has to be interpreted in the context of the MS(hydroxyalkoxyl) as referring to the hydroxyalkoxyl groups as the constituting units of hydroxyalkoxyl substituents, which either comprise a single hydroxyalkoxyl group or a side chain as outlined above, wherein two or more hydroxyalkoxyl units are covalently bound to each other by ether bonding. Within this definition it is not important whether the terminal hydroxyl group of a hydroxyalkoxyl substituent is further alkylated or not; both alkylated and non-alkylated hydroxyalkoxyl substituents are included for the determination of MS(hydroxyalkoxyl). The esterified cellulose ether a) generally has a molar substitution of hydroxyalkoxyl groups of at least 0.05, preferably at least 0.08, more preferably at least 0.12, and most preferably at least 0.15. The degree of molar substitution is generally not more than 1.00, preferably not more than 0.90, more preferably not more than 0.70, and most preferably not more than 0.50.

The average number of hydroxyl groups substituted by alkoxyl groups, such as methoxyl groups, per anhydroglucose unit, is designated as the degree of substitution of alkoxyl groups, DS(alkoxyl). In the above-given definition of DS, the term “hydroxyl groups substituted by alkoxyl groups” is to be construed within the present invention to include not only alkylated hydroxyl groups directly bound to the carbon atoms of the cellulose backbone, but also alkylated hydroxyl groups of hydroxyalkoxyl substituents bound to the cellulose backbone. The esterified cellulose ether a) preferably has a DS(alkoxyl) of at least 1.0, more preferably at least 1.1, even more preferably at least 1.2, most preferably at least 1.4, and particularly at least 1.6. The DS(alkoxyl) is preferably not more than 2.5, more preferably not more than 2.4, even more preferably not more than 2.2, and most not more than 2.05.

Most preferably the esterified cellulose ether a) is an esterified hydroxypropyl methylcellulose having a DS(methoxyl) within the ranges indicated above for DS(alkoxyl) and an MS(hydroxypropoxyl) within the ranges indicated above for MS(hydroxyalkoxyl).

The esterified cellulose ether a) has aliphatic monovalent acyl groups and groups of the formula —C(O)—R—COOH.

The aliphatic monovalent acyl groups which are present in the esterified cellulose ether a) are preferably acetyl, propionyl, or butyryl, such as n-butyryl or i-butyryl. Preferred groups of the formulas —C(O)—R—COOH are —C(O)—CH₂—CH₂—COOH.

Specific examples of esterified cellulose ethers a) are hydroxypropyl methylcellulose acetate succinate (HPMCAS), hydroxypropyl cellulose acetate succinate (HPCAS), hydroxybutyl methyl cellulose propionate succinate (HBMCPrS), hydroxyethyl hydroxypropyl cellulose propionate succinate (HEHPCPrS); or methyl cellulose acetate succinate (MCAS). Hydroxypropyl methylcellulose acetate succinates (HPMCAS) are the most preferred esterified cellulose ethers a).

In the esterified cellulose ether a) the degree of neutralization of the groups —C(O)—R—COOH is not more than 0.4, preferably not more than 0.3, more preferably not more than 0.2, most preferably not more than 0.1, and particularly not more than 0.05 or even not more than 0.01. The degree of neutralization can even be essentially zero or only slightly above it, e.g. up to 10⁻³ or even only up to 10⁻⁴. The term “degree of neutralization” as used herein defines the ratio of deprotonated carboxylic groups over the sum of deprotonated and protonated carboxylic groups, i.e.,

Degree of neutralization=[—C(O)—R—COO⁻]/[—C(O)—R—COO⁻+—C(O)—R—COOH].

If the groups —C(O)—R—COOH are partially neutralized, the cation preferably is an ammonium cation, such as NH₄⁺ or an alkali metal ion, such as the sodium or potassium ion, more preferably the sodium ion.

The esterified cellulose ether a) in the composition of the present invention has aliphatic monovalent acyl groups and groups of the formula —C(O)—R—COOH, such that the total degree of ester substitution is from 0.03 to 0.70. The sum of i) the degree of substitution of aliphatic monovalent acyl groups and ii) the degree of substitution of groups of formula —C(O)—R—COOH, of which the degree of neutralization is not more than 0.4, is an essential feature of the esterified cellulose ether a). The total degree of ester substitution is at least 0.03, generally at least 0.07, preferably at least 0.10, more preferably at least 0.15, most preferably at least 0.20, and particularly at least 0.25. The total degree of ester substitution in the esterified cellulose ether a) is not more than 0.70, generally not more than 0.67, preferably up to 0.65, more preferably up to 0.60, and most preferably up to 0.55 or up to 0.50. In one aspect of the present invention esterified cellulose ethers a) having a total degree of ester substitution of from 0.10 to 0.65 and particularly from 0.20 to 0.60 are preferred. In another aspect of the present invention esterified cellulose ethers a) having a total degree of ester substitution of from 0.20 to 0.50 and particularly from 0.25 to 0.44 are preferred.

The esterified cellulose ethers a) generally have a degree of substitution of aliphatic monovalent acyl groups, such as acetyl, propionyl, or butyryl groups, of at least 0.03 or 0.05, preferably at least 0.10, more preferably at least 0.15, most preferably at least 0.20, and particularly at least 0.25 or at least 0.30. The esterified cellulose ethers generally have a degree of substitution of aliphatic monovalent acyl groups of up to 0.69, preferably up to 0.60, more preferably up to 0.55, most preferably up to 0.50, and particularly up to 0.45 or even only up to 0.40. The esterified cellulose ethers a) generally have a degree of substitution of groups of formula —C(O)—R—COOH, such as succinoyl, of at least 0.01, preferably at least 0.02, more preferably at least 0.05, and most preferably at least 0.10. The esterified cellulose ethers generally have a degree of substitution of groups of formula —C(O)—R—COOH of up to 0.65, preferably up to 0.60, more preferably up to 0.55, and most preferably up to 0.50 or up to 0.45. As indicated above, the degree of neutralization of the groups —C(O)—R—COOH is not more than 0.4.

Moreover, in the esterified cellulose ether a) the sum of i) the degree of substitution of aliphatic monovalent acyl groups and ii) the degree of substitution of groups of formula —C(O)—R—COOH and iii) the degree of substitution of alkoxyl groups, DS(alkoxyl), generally is not more than 2.60, preferably not more than 2.55, more preferably not more than 2.50, and most preferably not more than 2.45. The esterified cellulose ether a) generally has a sum of degrees of substitution of i) aliphatic monovalent acyl groups and ii) groups of formula —C(O)—R—COOH and iii) of alkoxyl groups of at least 1.7, preferably at least 1.9, and most preferably at least 2.1.

The content of the acetate and succinate ester groups is determined according to “Hypromellose Acetate Succinate”, United States Pharmacopeia and National Formulary, NF 29, pp. 1548-1550. Reported values are corrected for volatiles (determined as described in section “loss on drying” in the above HPMCAS monograph). The method may be used in analogue manner to determine the content of propionyl, butyryl and other ester groups.

The content of ether groups in the esterified cellulose ether is determined in the same manner as described for “Hypromellose”, United States Pharmacopeia and National Formulary, USP 35, pp 3467-3469.

The contents of ether and ester groups obtained by the above analyses are converted to DS and MS values of individual substituents according to the formulas below. The formulas may be used in analogue manner to determine the DS and MS of substituents of other cellulose ether esters.

$$\% cellulose backbone = 100 - ( \% MeO * \frac{M ( {OCH}_{3} ) - M ( OH )}{M ( {OCH}_{3} )} ) - ( \% HPO * \frac{M ( {OCH}_{2} CH ( OH ) {CH}_{3} ) - M ( OH )}{M ( {OCH}_{2} CH ( OH ) {CH}_{3} )} ) - ( \% Acetyl * \frac{M ( {COCH}_{3} ) - M ( H )}{M ( {COCH}_{3} )} ) - ( \% Succinoyl * \frac{M ( {COC}_{2} {H}_{4} COOH ) - M ( H )}{M ( {COC}_{2} {H}_{4} COOH )} )$$

$$DS ( Me ) = \frac{\frac{\% MeO}{M ( {OCH}_{3} )}}{\frac{\% cellulose backbone}{M ( AGU )}}$$

$$MS ( HP ) = \frac{\frac{\% HPO}{M ( HPO )}}{\frac{\% cellulose backbone}{M ( AGU )}}$$

$$DS ( Acetyl ) = \frac{\frac{\% Acetyl}{M ( Acetyl )}}{\frac{\% cellulose backbone}{M ( AGU )}}$$

$$DS ( Succinoyl ) = \frac{\frac{\% Succinoyl}{M ( Succinoyl )}}{\frac{\% cellulose backbone}{M ( AGU )}}$$

$$M ( MeO ) = M ( {OCH}_{3} ) = 31.03 Da$$

$$M ( HPO ) = M ( {OCH}_{2} CH ( OH ) {CH}_{3} ) = 75.09 Da$$

$$M ( Acetyl ) = M ( {COCH}_{3} ) = 43.04 Da$$

$$M ( Succinoyl ) = M ( {COC}_{2} {H}_{4} COOH ) = 101.08 Da$$

$$M ( AGU ) = 162.14 Da$$

$$M ( OH ) = 17.008 Da$$

$$M ( H ) = 1.008 Da$$

By convention, the weight percent is an average weight percentage based on the total weight of the cellulose repeat unit, including all substituents. The content of the methoxyl group is reported based on the mass of the methoxyl group (i.e., —OCH₃). The content of the hydroxyalkoxyl group is reported based on the mass of the hydroxyalkoxyl group (i.e., —O— alkylene-OH); such as hydroxypropoxyl (i.e., —O—CH₂CH(CH₃)—OH). The content of the aliphatic monovalent acyl groups is reported based on the mass of —C(O)—R₁ wherein R₁ is a monovalent aliphatic group, such as acetyl (—C(O)—CH₃). The content of the group of formula —C(O)—R—COOH is reported based on the mass of this group, such as the mass of succinoyl groups (i.e., —C(O)—CH₂—CH₂—COOH).

Another essential property of the esterified cellulose ether a) is its water-solubility. The esterified cellulose ether generally has a solubility in water of at least 2.0 weight percent at 2° C., i.e., it can be dissolved as an at least 2.0 weight percent solution, preferably at least 3.0 weight percent solution, more preferably at least 5.0 weight percent solution or even at least 10.0 weight solution in water at 2° C. Generally the esterified cellulose ether a) can be dissolved as up to 20 weight percent solution or in the most preferred embodiments even as up to 30 weight percent solution in water at a temperature of 2° C. The term “an x weight percent solution in water at 2° C.” as used herein means that x g of the esterified cellulose ether b) is soluble in (100−x) g of water at 2° C.

In more general terms, the esterified cellulose ether a), in spite of its low degree of neutralization of the groups —C(O)—R—COOH, is soluble in an aqueous liquid at a temperature of less than 10° C., more preferably less than 8° C., even more preferably 5° C. or less, and most preferably up to 3° C., even when the esterified cellulose ether is blended with an aqueous liquid that does not increase the degree of neutralization of the esterified cellulose ether a) to more than 0.4 or a preferred range listed above, e.g., when the esterified cellulose ether is blended with only water, such as deionized or distilled water. Clear or turbid solutions with only a small portion of sediment or in the preferred embodiments even without sediment are obtained at 2° C. When the temperature of the prepared solution is increased to 20° C., no precipitation occurs.

The esterified cellulose ether a) comprised in the composition of the present invention generally has a viscosity of at least 1.2 mPa·s, preferably least 1.8 mPa·s, and more preferably least 2.4 mPa·s, and generally no more than 200 mPa·s, preferably no more than 100 mPa·s, more preferably no more than 50 mPa·s, and most preferably no more than 30 mPa·s, measured as a 2.0 weight percent solution of the esterified cellulose ether in 0.43 wt. % aqueous NaOH at 20° C. according to “Hypromellose Acetate Succinate, United States Pharmacopia and National Formulary, NF 29, pp. 1548-1550”.

The esterified cellulose ether a) generally has a weight average molecular weight M_{w} of up to 500,000 Dalton, preferably up to 250,000 Dalton, more preferably up to 200,000 Dalton, and most preferably up to 150,000 Dalton. Generally it has a weight average molecular weight M_{w} of at least 10,000 Dalton, preferably at least 15,000 Dalton, more preferably at least 20,000 Dalton, and most preferably at least 30,000 Dalton. M_{w} and the number average molecular weight Mₙ are measured according to Journal of Pharmaceutical and Biomedical Analysis 56 (2011) 743 using a mixture of 40 parts by volume of acetonitrile and 60 parts by volume of aqueous buffer containing 50 mM NaH₂PO₄ and 0.1 M NaNO₃ as mobile phase. The mobile phase is adjusted to a pH of 8.0. The measurement of M_{w} and Mₙ is described in more details in the Examples.

The production of the esterified cellulose ether a) is described in copending International Patent Application WO 2016/148977, filed Mar. 8, 2016, which claims the priority of U.S. Provisional Application 62/133,514, filed Mar. 16, 2015 and International Patent Application WO 2016/148976, filed Mar. 8, 2016, which claims the priority of U.S. Provisional Application No. 62/133,518, filed on 16 Mar. 2015, all filed by the Applicants of the present patent application, and in the Examples of the present invention. These International Patent Applications describe the reaction of a cellulose ether with an aliphatic monocarboxylic acid anhydride, such as acetic anhydride, butyric anhydride or propionic anhydride, and with a dicarboxylic acid anhydride, such as succinic anhydride, in an aliphatic carboxylic acid, such as acetic acid, as a reaction diluent.

In the International Patent Application WO 2016/148977, filed Mar. 8, 2016, which claims the priority of U.S. Provisional Application No. 62/133,514, the esterified cellulose ether a) is produced in the absence of an esterification catalyst, and in particular in the absence of an alkali metal carboxylate. This is in contrast to known processes. According to the general procedure described in the International Patent Application WO 2016/148977, a cellulose ether, preferably one of the type listed further above, is reacted with an aliphatic monocarboxylic acid anhydride, such as acetic anhydride, butyric anhydride and propionic anhydride, and with a dicarboxylic acid anhydride, such as succinic anhydride. The molar ratio between the anhydride of an aliphatic monocarboxylic acid and the anhydroglucose units of the cellulose ether generally is from 0.1/1 to 7/1, preferably from 0.3/1 to 3.5/1, and more preferably from 0.5/1 to 2.5/1. The molar ratio between the anhydride of a dicarboxylic acid and the anhydroglucose units of cellulose ether generally is from 0.1/1 to 2.2/1, preferably from 0.2/1 to 1.2/1, and more preferably from 0.3/1 to 0.8. The molar number of anhydroglucose units of the cellulose ether can be determined from the weight of the cellulose ether used as a starting material, by calculating the average molecular weight of the substituted anhydroglucose units from the DS(alkoxyl) and MS(hydroxyalkoxyl). The esterification of the cellulose ether is conducted in an aliphatic carboxylic acid as a reaction diluent, such as acetic acid, propionic acid, or butyric acid, most preferably acetic acid. The molar ratio [aliphatic carboxylic acid/anhydroglucose units of cellulose ether] generally is at least 0.7/1, preferably at least 1.2/1, and more preferably at least 1.5/1. The molar ratio [aliphatic carboxylic acid/anhydroglucose units of cellulose ether] is generally up to 10/1, and preferably up to 9/1. Lower ratios, such as up to 7/1 or even only up to 4/1 and under optimized conditions even only up to 2/1 can also be used, which makes optimal use of the amount of reaction diluent needed. In contrast to the known processes, the esterified cellulose ethers of the present invention are produced in the absence of an esterification catalyst, and in particular in the absence of a alkali metal carboxylate. The reaction temperature for the esterification is generally from 60° C. to 110° C., preferably from 70° C. to 100° C. The esterification reaction is typically completed within 2 to 8 hours, more typically within 3 to 6 hours. After completion of the esterification reaction, the esterified cellulose ether can be precipitated from the reaction mixture in a known manner, for example as described in U.S. Pat. No. 4,226,981, International Patent Application WO 2005/115330, European Patent Application EP 0 219 426 or International Patent Application WO2013/148154. The precipitated esterified cellulose ether is subsequently washed with water, preferably at a temperature of from 70 to 100° C.

Moreover, the composition of the present invention comprises a cellulose ether having a viscosity of from 1.2 to 200 mPa·s, preferably from 1.8 to 100 mPa·s, more preferably from 2.4 to 50 mPa·s and in particular from 2.8 to 5.0 mPa·s, measured as a 2 weight-% solution in water at 20° C. The 2% by weight cellulose ether solution in water is prepared according to United States Pharmacopeia (USP 35, “Hypromellose”, pages 3467-3469) followed by an Ubbelohde viscosity measurement according to DIN 51562-1:1999-01 (January 1999).

The cellulose ether is generally non-ionic and water-soluble. A water-soluble cellulose ether is a cellulose ether that has a solubility in water of at least 2 grams in 100 grams of distilled water at 25° C. and 1 atmosphere. The non-ionic cellulose ether preferably is a hydroxyalkyl alkylcellulose or an alkylcellulose. Nonlimiting examples of non-ionic water soluble cellulose ethers include C₁-C₃-alkyl celluloses, such as methylcelluloses; C₁-C₃-alkyl hydroxy-C₁₋₃-alkyl celluloses, such as hydroxyethyl methylcelluloses, hydroxypropyl methylcelluloses or ethyl hydroxyethyl celluloses; hydroxy-C₁₋₃-alkyl celluloses, such as hydroxyethyl celluloses or hydroxypropyl celluloses; mixed hydroxy-C₁-C₃-alkyl celluloses, such as hydroxyethyl hydroxypropyl celluloses, mixed C₁-C₃-alkyl celluloses, such as methyl ethyl celluloses, or ternary cellulose ethers, such as ethyl hydroxypropyl methyl celluloses, ethyl hydroxyethyl methyl celluloses, hydroxyethyl hydroxypropyl methyl celluloses, or alkoxy hydroxyethyl hydroxypropyl celluloses, the alkoxy group being straight-chain or branched and containing 2 to 8 carbon atoms.

In an embodiment, the cellulose ether is methylcellulose, hydroxyethyl cellulose, hydroxypropyl cellulose, hydroxyethyl methylcellulose, hydroxypropyl methylcellulose, hydroxybutyl methylcellulose, or ethylhydroxyethyl cellulose. Preferably the cellulose ether is a methycellulose (MC) or, more preferably, a hydroxyalkyl alkylcellulose, such as hydroxypropyl methylcellulose (HPMC).

The cellulose ether preferably has a DS(alkyl) of from 1.0 to 2.5, more preferably from 1.1 to 2.4, most preferably from 1.5 to 2.2, and particularly from 1.6 to 2.05. The degree of the alkyl substitution, DS(alkyl), of a cellulose ether is the average number of OH groups substituted with alkyl groups, preferably methyl groups, per anhydroglucose unit. For determining the DS(alkyl), the term “OH groups substituted with alkyl groups” does not only include the alkylated OH groups directly bound to the carbon atoms of the cellulose backbone but also alkylated OH groups that have been formed after hydroxyalkylation.

The cellulose ether generally has an MS(hydroxyalkyl) of 0 to 1.10, preferably 0.05 to 0.90, more preferably 0.12 to 0.75, most preferably 0.15 to 0.60, and particularly 0.21 to 0.50. The degree of the hydroxyalkyl substitution is described by the MS (molar substitution). The MS(hydroxyalkyl) is the average number of hydroxyalkyl groups which are bound by an ether bond per mole of anhydroglucose unit. During the hydroxyalkylation, multiple substitutions can result in side chains.

The term “hydroxyl group substituted with alkyl group” or “hydroxyl group substituted with hydroxyalkyl group” as used herein means that the hydrogen atom on the hydroxyl group is replaced by an alkyl group or a hydroxyalkyl group.

The sum of the MS(hydroxyalkyl) and the DS(alkyl) preferably is at least 1.5, more preferably at least 1.7, most preferably at least 1.9, and preferably up to 2.9, or up to 2.7, or up to 2.5.

The determination of the % methoxyl in methylcellulose (MC) is carried out according to the United States Pharmacopeia (USP35, “Methylcellulose”, pages 3868-3869). The determination of the % methoxyl and % hydroxypropoxyl in hydroxypropyl methylcellulose (HPMC) is carried out according to the United States Pharmacopeia (USP 35, “Hypromellose”, pages 3467-3469). The values obtained as % methoxyl and % hydroxypropoxyl are subsequently converted into degree of substitution (DS) for methyl substituents and molar substitution (MS) for hydroxypropyl substituents. Residual amounts of salt are taken into account in the conversion. Based on these methods, the skilled artisans know how to determine MS(hydroxyalkyl) and DS(alkyl) of other cellulose ethers.

The determination of the ether substitution of other ethers than methylcellulose and hydroxypropyl methylcellulose, such as hydroxyethyl methylcellulose (HEMC), can be effected as described by K. L. Ketterer, W. E. Kester, D. L. Wiederrich, and J. A. Grover, Determination of Alkoxyl Substitution in Cellulose Ethers by Zeisel-Gas Chromatographie, Analytical Chemistry, Vol. 51, No. 13, November 1979, 2172-76.

The composition of the present invention preferably comprises from 5 to 95 percent, more preferably from 15 to 85 percent, and most preferably from 25 to 75 percent of the esterified cellulose ether a) and from 95 to 5 percent, more preferably from 85 to 15 percent, and most preferably from 75 to 25 percent of the cellulose ether b) as described above, based on the total weight of components a) and b).

The composition of the present invention preferably is in the form of an aqueous solution. The aqueous solution may comprise a minor amount of one or more organic solvents; however, the aqueous solution should generally comprise at least 80 percent, preferably at least 85 percent, more preferably at least at least 90 percent, and particularly at least 95 percent of water, based on the total weight of water and the organic solvent. Preferred organic liquid diluents are polar organic solvents having one or more heteroatoms, such as oxygen, nitrogen or halogen like chlorine. More preferred organic liquid diluents are alcohols, for example multifunctional alcohols, such as glycerol, or preferably monofunctional alcohols, such as methanol, ethanol, isopropanol or n-propanol; ethers, such as tetrahydrofuran, ketones, such as acetone, methyl ethyl ketone, or methyl isobutyl ketone; acetates, such as ethyl acetate; halogenated hydrocarbons, such as methylene chloride; or nitriles, such as acetonitrile. More preferably the organic liquid diluents have 1 to 6, most preferably 1 to 4 carbon atoms. The composition of the present invention may comprise a basic compound, but the degree of neutralization of the groups —C(O)—R—COOH of the esterified cellulose ether a) in the composition of the present invention should not be more than 0.4, preferably not more than 0.3 or 0.2 or 0.1, more preferably not more than 0.05 or 0.01, and most preferably not more than 10⁻³ or even not more than 10⁻⁴. Preferably the composition of the present invention does not comprise a substantial amount of a basic compound. More preferably, the composition of the present invention does not contain a basic compound. Preferably the aqueous composition of the present invention comprises only water as a diluent, in the absence of an organic solvent.

The composition of the present invention preferably comprises at least 0.2 wt.-%, more preferably at least 0.5 wt.-%, and most preferably at least 1.0 wt.-%, and preferably up to 20 wt.-%, more preferably up to 15 wt.-%, and most preferably up to 10 wt.-%, of an esterified cellulose ether a), based on the total weight of the composition of the present invention. The composition of the present invention preferably comprises at least 0.2 wt.-%, more preferably at least 0.5 wt.-%, and most preferably at least 1.0 wt.-%, and preferably up to 15 wt.-%, more preferably up to 10 wt.-%, and most preferably up to 5 wt.-%, of a cellulose ether a), based on the total weight of the composition.

The composition of the present invention may further comprise one or more active ingredients, such as one or more drugs, and/or one or more optional adjuvants, such as coloring agents, pigments, opacifiers, flavor and taste improvers, antioxidants, and any combination thereof. The term “drug” is conventional, denoting a compound having beneficial prophylactic and/or therapeutic properties when administered to an animal, especially humans

The esterified cellulose ether a) and the cellulose ether b) can be brought into aqueous solution by cooling the aqueous composition to a temperature of −2° C. to less than 10° C., preferably of 0° C. to less than 8° C., more preferably of 0.5° C. to less than 5° C., and most preferably of 0.5° C. to 3° C. When the temperature of the prepared aqueous solution is increased to 20° C., no precipitation occurs. The aqueous solution gels at slightly elevated temperature, typically at 30 to 55° C. A gel formed from an aqueous solution comprising the above-mentioned cellulose ether b) in addition to the above-mentioned esterified cellulose ether a) displays reduced or even no syneresis, even when the temperature of the gel is further increased, for example to a temperature above 60° C., or even to 70° C. or more, and generally up to 90° C., typically up to 85° C. A comparative composition which only comprises the esterified cellulose ether a) typically displays a higher degree of syneresis than a comparable composition of the present invention when the temperature of the gel is increased to a temperature above 60° C., or even to 70° C. or more, and generally up to 90° C., typically up to 85° C. The reduced or lacking syneresis of the composition of the present invention is very useful in applications where heating to a temperature of more than about 55° C., typically more than about 60° C., or even to 70° C. or more is desired. For example in the production of polymeric capsule shells, hot dipping pins can be dipped into the aqueous solution of the esterified cellulose ether a) and the cellulose ether b) and gelation of the solution can be effected to produce a film on the hot dipping pins without film breakage caused by syneresis. Also reduced or lacking syneresis of the composition of the present invention enables high drying temperatures of the films produced from the composition without film breakage. The possibility of reducing or even avoiding syneresis, i.e., reducing or avoiding expulsion of water from the gelled composition of the present invention, increases the processing window of the composition of the present invention.

Even more surprisingly, it has been found that the incorporation of the above-mentioned cellulose ether b) into the aqueous solution comprising the above-mentioned esterified cellulose ether a) does not reduce the storage modulus or gel strength of a gel formed from such aqueous solution to an undue degree.

The aqueous composition of the present invention is particularly useful in the manufacture of capsules which comprises the step of contacting the aqueous composition with dipping pins. Partial neutralization of the esterified cellulose ether, which might impact the enteric properties of the esterified cellulose ether, is not needed. Typically an aqueous composition having a temperature of less than 23° C., more typically less than 15° C. or in some embodiments less than 10° C. is contacted with dipping pins that have a higher temperature than the aqueous composition and that have a temperature of at least 21° C., typically at least 30° C., and more typically at least 50° C. and generally up to 95° C., preferably up to 85° C., and more preferably up to 75° C. The capsules have enteric properties. The aqueous composition of the present invention is also useful for coating dosage forms, such as tablets, granules, pellets, caplets, lozenges, suppositories, pessaries or implantable dosage forms.

Some embodiments of the invention will now be described in detail in the following Examples.

### EXAMPLES

Unless otherwise mentioned, all parts and percentages are by weight. In the Examples the following test procedures are used.

Hydroxypropyl Methyl Cellulose (HPMC)

The content of ether groups in HPMC is determined as described for “Hypromellose”, United States Pharmacopeia and National Formulary, USP 35, pp 3467-3469.

The viscosity of the HPMC is measured as a 2.0% by weight solution in water at 20° C.±0.1° C. The 2.0% by weight HPMC solution in water is prepared according to United States Pharmacopeia (USP 35, “Hypromellose”, pages 3467-3469), followed by an Ubbelohde viscosity measurement according to DIN 51562-1:1999-01 (January 1999).

Hydroxypropyl Methyl Cellulose Acetate Succinate (HPMCAS)

The content of ether groups in the HPMCAS is determined in the same manner as described for “Hypromellose”, United States Pharmacopeia and National Formulary, USP 35, pp 3467-3469.

The ester substitution with acetyl groups (—CO—CH₃) and the ester substitution with succinoyl groups (—CO—CH₂—CH₂—COOH) are determined according to Hypromellose Acetate Succinate, United States Pharmacopia and National Formulary, NF 29, pp. 1548-1550”. Reported values for ester substitution are corrected for volatiles (determined as described in section “loss on drying” in the above HPMCAS monograph).

M_{w} and Mₙ of HPMCAS are measured according to Journal of Pharmaceutical and Biomedical Analysis 56 (2011) 743 unless stated otherwise. The mobile phase is a mixture of 40 parts by volume of acetonitrile and 60 parts by volume of aqueous buffer containing 50 mM NaH₂PO₄ and 0.1 M NaNO₃. The mobile phase is adjusted to a pH of 8.0. Solutions of the cellulose ether esters (HPMCAS) are filtered into a HPLC vial through a syringe filter of 0.45 μm pore size. The exact details of measuring M_{w} and Mₙ are disclosed in the International Patent Application No. WO 2014/137777 in the section “Examples” under the title “Determination of M_{w}, Mₙ and M_{z}”. Except for HPMCAS Sample II, the recovery rate of all HPMCAS samples is at least 97%.

Water-Solubility of HPMCAS

A 2 wt. percent mixture of HPMCAS and water is prepared by mixing 2.0 g HPMCAS, based on its dry weight, with 98.0 g water under vigorous stirring at 0.5° C. for 16 hours. The temperature of the mixture of HPMCAS and water is then increased to 5° C. The water solubility of the esterified cellulose ether is determined by visual inspection. The determination whether the HPMCAS is water-soluble at 2% at 5° C. or not is done as follows. “Water soluble at 2% —yes” means that a solution without sediment is obtained according to the procedure above.

Storage Modulus of Aqueous Solutions of HPMCAS and Optionally HPMC

A solution of HPMCAS and optionally HPMC in water is produced by adding, dried HPMCAS and optionally HPMC (under consideration of the water content of the HPMCAS and HPMC) to water (temperature 20-25° C.) at the desired concentrations at room temperature while stirring with an overhead lab stirrer at 750 rpm with a 3-wing (wing=2 cm) blade stirrer. The solution is then cooled to about 1.5° C. After the temperature of 1.5° C. is reached the solution is stirred for 120 min at 500 rpms. Each solution is stored in the refrigerator prior to the characterization.

Rheology measurements of the solutions of the HPMCAS and optionally HPMC in water are conducted with a Haake RS600 (Thermo Fisher Scientific) rheormeter with cup and bob fixtures (CC-25). The sample is heated at a rate of 1° C. per minute over a temperature range from 5 to 85° C. with a constant strain (deformation) of 2% and a constant angular frequency of 2 Hz. The measurement collection rate is chosen to be 4 data points/min. The storage modulus G′, which is obtained from the rheology measurements, represents the elastic properties of the solution and represents the gel strength in the high temperature region, when the storage modulus G′ is higher as the loss modulus G″.

Production of the HPMCAS Samples I-IV

The water-soluble HPMCAS polymer is produced as described in co-pending International Patent Application WO 2016/148977, filed Mar. 8, 2016, claiming the priority of U.S. Provisional Application 62/133,514, filed Mar. 16, 2015.

Succinic anhydride and acetic anhydride are dissolved at 70° C. in glacial acetic acid. Then hydroxypropyl methyl cellulose (HPMC, water free) is added under stirring. The amounts are listed in Table 1 below. The amount of HPMC is calculated on a dried basis. No amount of sodium acetate is added.

The HPMC has a methoxyl substitution (DS_{M}) of 1.92, a hydroxypropoxyl substitution (MS_{H}P) of 0.24 and a viscosity of 3.0 mPa·s, measured as a 2% solution in water at 20° C. The weight average molecular weight of the HPMC is about 20,000 Dalton. The HPMC is commercially available from The Dow Chemical Company as Methocel E3 LV Premium cellulose ether.

Then the reaction mixture is heated up to 85-110° C. for 2-3 hours until the desired substitution with acetyl groups and succinoyl groups is achieved. Then the crude product is precipitated by adding 1-2 L of water having a temperature of 21° C. Subsequently the precipitated product is separated from the mixture by filtration and washed several times with water having the temperature listed in Table 1 below. Then the product is isolated by filtration and dried at 55° C. overnight.

The properties of the water-soluble HPMCAS samples are listed in Table 2 below. In Table 2 the abbreviations have the following meanings:

DS_{M}=DS(methoxyl): degree of substitution with methoxyl groups;
MS_{HP}=MS(hydroxypropoxyl): molar subst. with hydroxypropoxyl groups;
DS_{Ac}: degree of substitution of acetyl groups;
DSₛ: degree of substitution of succinoyl groups.

TABLE 1

| | | | Glacial acetic | | | | | | | | |
|---|---|---|---|---|---|---|---|---|---|---|---|
| water-soluble | | | acid | | Succinic anhydride | | Acetic anhydride | | Sodium acetate | | Temperature |
| HPMCAS | HPMC* | | | mol/mol | | mol/mol | | mol/mol | | mol/mol | of washing |
| sample | g | Mol | g | HPMC | g | HPMC | g | HPMC | g | HPMC | water, ° C. |
| I | 350 | 1.72 | 448.7 | 4.25 | 89.7 | 0.52 | 269.2 | 1.59 | 0 | 0 | 95 |
| II | 350 | 1.72 | 717.9 | 6.8 | 62.8 | 0.36 | 323.1 | 1.91 | 0 | 0 | 95 |
| III | 350 | 1.72 | 179.5 | 1.7 | 179.5 | 1.04 | 538.5 | 3.18 | 0 | 0 | 95 |
| IV | 195 | 0.96 | 100 | 1.7 | 50 | 0.52 | 150 | 1.59 | 0 | 0 | 95 |

TABLE 2

| water-soluble | Molecular | | | | | | | | | | Sum | Water- |
|---|---|---|---|---|---|---|---|---|---|---|---|---|
| HPMCAS | weight (kDA) | | Methoxyl | Hydroxy- | Acetyl | Succinoyl | | | | | DS_{Ac} + | soluble |
| sample | Mₙ | M_{w} | (%) | propoxyl (%) | (%) | (%) | DS_{M} | MS_{HP} | DS_{Ac} | DSₛ | DSₛ | at 2% |
| I | 25 | 76 | 25.8 | 8.2 | 8.0 | 4.9 | 1.94 | 0.26 | 0.43 | 0.11 | 0.54 | Yes |
| II | *) | *) | 25.8 | 8.1 | 11.3 | 2.3 | 1.95 | 0.25 | 0.62 | 0.05 | 0.67 | Yes |
| III | 31 | 119 | 26.4 | 8.2 | 5.7 | 5.3 | 1.93 | 0.25 | 0.3 | 0.12 | 0.42 | Yes |
| IV | 23 | 57 | 24.8 | 7.7 | 3.2 | 11.4 | 1.89 | 0.24 | 0.18 | 0.27 | 0.45 | Yes |

*) Insufficient recovery

### Aqueous Solutions of HPMCAS and Optionally HPMC

Solutions of a HPMCAS and optionally a HPMC in water are prepared. The type and concentration of the HPMCAS sample is listed in Table 3 below. The HPMC is commercially available from The Dow Chemical Company as Methocel E3 LV Premium cellulose ether and has a methoxyl substitution (DS_{M}) of 1.92, a hydroxypropoxyl substitution (MS_{HP}) of 0.24 and a viscosity of 3.0 mPa·s, measured as a 2% solution in water at 20° C. The aqueous solutions are prepared as described above in the paragraph “Storage Modulus of Aqueous Solutions of HPMCAS and optionally HPMC”.

TABLE 3

| (Comparative) | % total | | |
|---|---|---|---|
| Example | polymer ¹⁾ | % HPMCAS ¹⁾ | % HPMC ¹⁾ |
| Ex. 1 | 2.0% | 1.4% HPMCAS-I | 0.6% |
| Ex. 2 | 5.0% | 3.5% HPMCAS-I | 1.5% |
| Ex. 3 | 5.0% | 2.5% HPMCAS-I | 2.5% |
| Ex. 4 | 5.0% | 1.5% HPMCAS-I | 3.5% |
| Comp. Ex. A | 2.0% | 2.0% HPMCAS-I | — |
| Comp. Ex. B | 5.0% | 5.0% HPMCAS-I | — |
| Ex. 5 | 2.0% | 1.4% HPMCAS-II | 0.6% |
| Ex. 6 | 2.0% | 1.0% HPMCAS-II | 1.0% |
| Ex. 7 | 5.0% | 3.5% HPMCAS-II | 1.5% |
| Ex. 8 | 5.0% | 2.5% HPMCAS-II | 2.5% |
| Ex. 9 | 5.0% | 1.5% HPMCAS-II | 3.5% |
| Comp. Ex. C | 2.0% | 2.0% HPMCAS-II | — |
| Comp. Ex. D | 5.0% | 5.0% HPMCAS-II | — |
| Ex. 10 | 2.0% | 1.4% HPMCAS-III | 0.6% |
| Ex. 11 | 5.0% | 3.5% HPMCAS-III | 1.5% |
| Ex. 12 | 5.0% | 2.5% HPMCAS-III | 2.5% |
| Ex. 13 | 5.0% | 1.5% HPMCAS-III | 3.5% |
| Comp. Ex. E | 2.0% | 2.0% HPMCAS-III | — |
| Comp. Ex. F | 5.0% | 5.0% HPMCAS-III | — |
| Ex. 14 | 2.0% | 1.4% HPMCAS-IV | 0.6% |
| Ex. 15 | 2.0% | 1.0% HPMCAS-IV | 1.0% |
| Ex. 16 | 5.0% | 3.5% HPMCAS-IV | 1.5% |
| Ex. 17 | 5.0% | 2.5% HPMCAS-IV | 2.5% |
| Ex. 18 | 5.0% | 1.5% HPMCAS-IV | 3.5% |
| Comp. Ex. G | 2.0% | 2.0% HPMCAS-IV | — |
| Comp. Ex. H | 5.0% | 5.0% HPMCAS-IV | — |

¹⁾ based on total weight of aqueous solution

Rheology measurements of the aqueous solutions of Examples 1-18 and Comparative Examples A-H are carried out to measure the storage modulus G′ as a function of temperature. The storage modulus G′, which is obtained from the rheology measurements, represents the elastic properties of the solution and represents the gel strength in the high temperature region, when the storage modulus G′ is higher than the loss modulus G″.

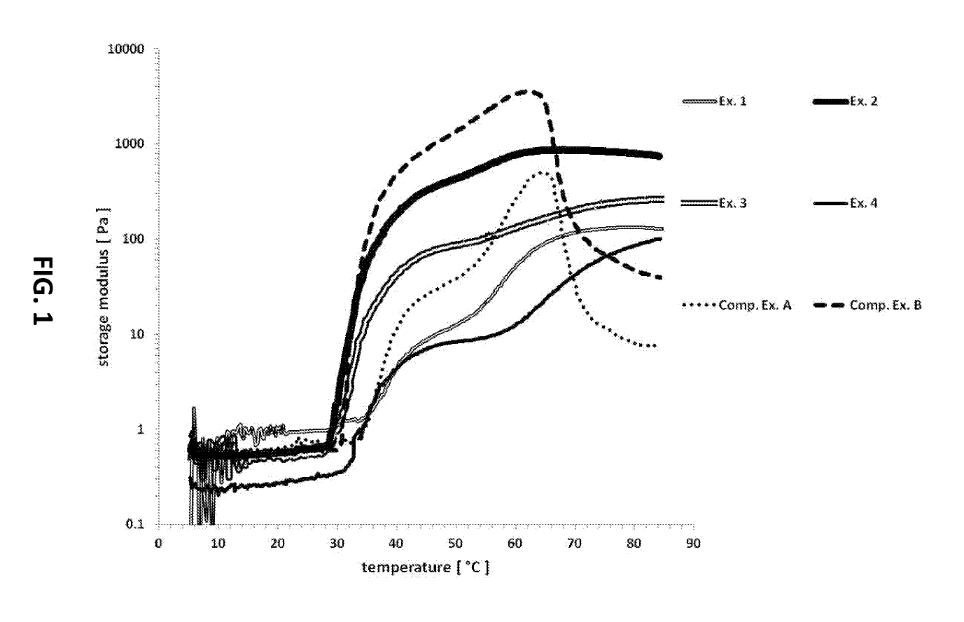

The storage modulus G′ as a function of temperature of the aqueous compositions of Examples 1-4 and Comparative Examples A and B is illustrated in FIG. 1.

Comparative Example A (2.0% HPMCAS-I) exhibits a high storage modulus G′ (gel strength) at mildly elevated temperatures of up to about 65° C. However, at a temperature above about 65° C., the storage modulus G′ breaks down due to syneresis of the gel. The same observation is made for the aqueous composition of Comparative Example B (5.0% HPMCAS-I).

The maximum gel strengths of the aqueous compositions of Examples 1-4 are not quite as high as those of Comparative Examples A and B, but at temperatures above 65° C. no significant reduction in storage modulus G′ is observed.

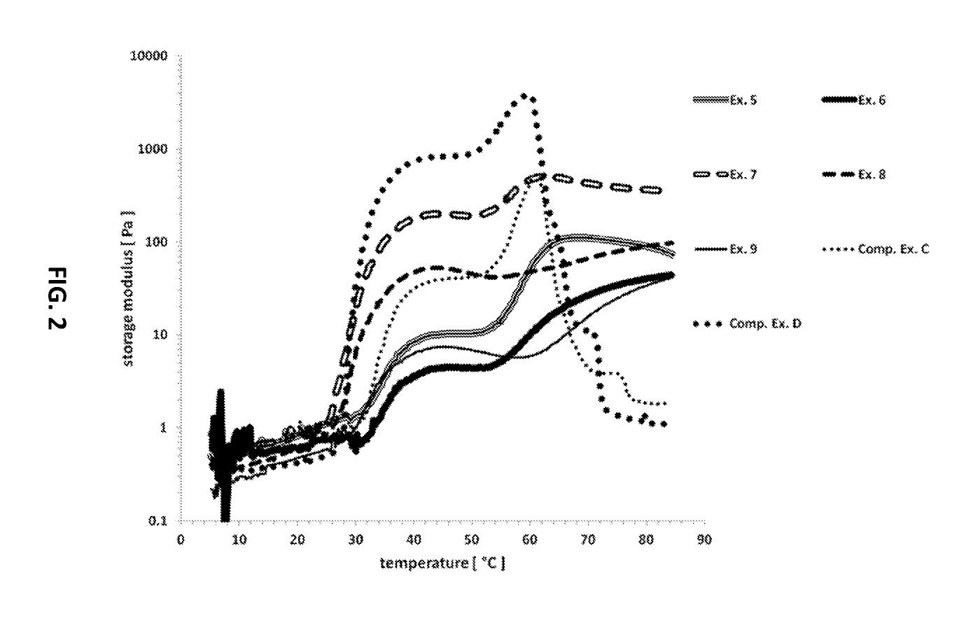

Very similar observations are made for the compositions of Comparative Examples C and D and of Examples 5-9 which are illustrated in FIG. 2. At a temperature above about 60° C., the storage modulus G′ of the aqueous compositions of Examples B and C breaks down due to syneresis of the gels. The maximum gel strengths of the aqueous compositions of Examples 5-9 are not quite as high as those of Comparative Examples C and D, but at temperatures above 65° C. no significant reduction in storage modulus G′ is observed.

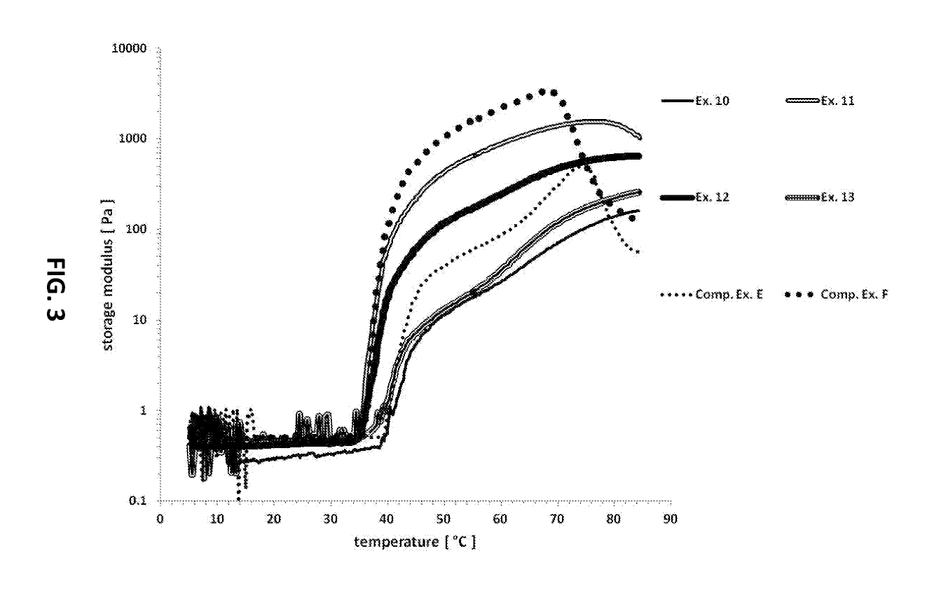

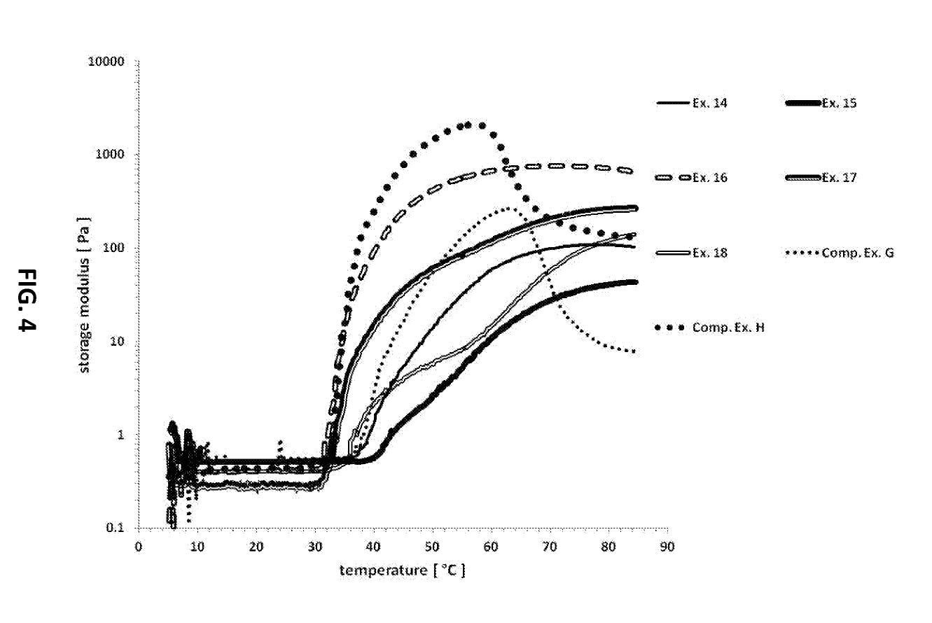

Again very similar observations are made for the compositions of Comparative Examples E and F and of Examples 10-13 which are illustrated in FIG. 4 and for the compositions of Comparative Examples G and H and of Examples 14-18 which are illustrated in FIG. 3.

Gelation

Aqueous solutions of the Examples and Comparative Examples as listed in Table 4 below were gelled by heating the aqueous solutions in a glass bottle to a temperature as listed in Table 4 below for 60 min.

The degree of syneresis is assessed by visual inspection and given the following ratings:

1: No visible syneresis; a glass bottle containing the gelled aqueous solution can be turned upside down without causing the gel to flow. In the bottle that has been turned upside down the gel stays on top and does not flow down.

2: Small amount of water is visibly expulsed. When a glass bottle containing the gelled aqueous solution is turned upside down, the gel mass does not stay on top but falls down to the bottom because the volume of the gel somewhat shrinks due to water expulsion from the gel.

3: Larger amount of water is visibly expulsed than at rating 2; gravitation behavior of gel as in rating 2; volume of the gel clearly shrinks due to water expulsion from the gel.

4: Larger amount of water is visibly expulsed than at rating 3; volume of expulsed liquid is larger than the volume of remaining gel; volume of gel shrinks to a significant degree due to water expulsion from the gel.

5: Larger amount of water is visibly expulsed than at rating 4; volume of expulsed liquid is significantly larger than the volume of remaining gel; volume of gel shrinks to a high degree due to water expulsion from the gel.

6: Larger amount of water is visibly expulsed than at rating 5; volume of expulsed liquid is much larger than the volume of remaining gel; volume of gel shrinks to a high very degree due to water expulsion from the gel.

TABLE 4

| | | | Heating | |
|---|---|---|---|---|
| | | | temper- | Rating of |
| (Comparative) | | | ature | Visual |
| Example | % HPMCAS ¹⁾ | % HPMC ¹⁾ | (° C.) | Inspection |
| Ex. 10 | 1.4% HPMCAS-III | 0.6% | 40° C. | 1 |
| Ex. 11 | 3.5% HPMCAS-III | 1.5% | 40° C. | 1 |
| Comp. Ex. E | 2.0% HPMCAS-III | — | 40° C. | 1 |
| Comp. Ex. F | 5.0% HPMCAS-III | — | 40° C. | 1 |
| Ex. 10 | 1.4% HPMCAS-III | 0.6% | 60° C. | 1 |
| Ex. 11 | 3.5% HPMCAS-III | 1.5% | 60° C. | 1 |
| Comp. Ex. E | 2.0% HPMCAS-III | — | 60° C. | 1 |
| Comp. Ex. F | 5.0% HPMCAS-III | — | 60° C. | 2 |
| Ex. 10 | 1.4% HPMCAS-III | 0.6% | 70° C. | 3 |
| Ex. 11 | 3.5% HPMCAS-III | 1.5% | 70° C. | 2 |
| Comp. Ex. E | 2.0% HPMCAS-III | — | 70° C. | 4 |
| Comp. Ex. F | 5.0% HPMCAS-III | — | 70° C. | 4 |
| Ex. 10 | 1.4% HPMCAS-III | 0.6% | 80° C. | 3-4 |
| Ex. 11 | 3.5% HPMCAS-III | 1.5% | 80° C. | 3 |
| Comp. Ex. E | 2.0% HPMCAS-III | — | 80° C. | 5 |
| Comp. Ex. F | 5.0% HPMCAS-III | — | 80° C. | 5 |
| Ex. 5 | 1.4% HPMCAS-II | 0.6% | 80° C. | 5 |
| Ex. 7 | 3.5% HPMCAS-II | 1.5% | 80° C. | 3-4 |
| Comp. Ex. C | 2.0% HPMCAS-II | — | 80° C. | 6 |
| Comp. Ex. D | 5.0% HPMCAS-II | — | 80° C. | 6 |

¹⁾ based on total weight of aqueous solution

1. A composition in the form of an aqueous solution or a gel comprising
a) an esterified cellulose ether comprising aliphatic monovalent acyl groups and groups of the formula —C(O)—R—COOH, R being a divalent hydrocarbon group, wherein I) the degree of neutralization of the groups —C(O)—R—COOH is not more than 0.4 and
II) the total degree of ester substitution is from 0.03 to 0.70, and
b) a cellulose ether having a viscosity of from 1.2 to 200 mPa·s, measured as a 2 weight-% aqueous solution at 20° C.

2. The composition of claim 1 wherein the total degree of ester substitution in component a) is from 0.20 to 0.60.

3. The composition of claim 1 wherein in component a) the aliphatic monovalent acyl groups are acetyl, propionyl or butyryl groups, and the groups of the formula —C(O)—R—COOH are —C(O)—CH₂—CH₂—COOH groups.

4. The composition of claim 1 wherein component a) is an esterified hydroxyalkyl alkylcellulose.

5. The composition of claim 1 wherein component a) is hydroxypropyl methylcellulose acetate succinate.

6. The composition of claim 1 wherein the esterified cellulose ether a) has a solubility in water of at least 2.0 weight percent at 2° C.

7. The composition of claim 1 wherein the cellulose ether b) has a viscosity of from 2.8 to 5.0 mPa·s, measured as a 2 weight-% aqueous solution at 20° C.

8. The composition of claim 1 wherein the cellulose ether b) is a hydroxyalkyl alkylcellulose.

9. The composition of claim 1 wherein the cellulose ether b) is hydroxypropyl methylcellulose.

10. The composition of claim 1 comprising from 15 to 85 percent of component a) and from 85 to 15 percent of component b), based on the total weight of components a) and b).

11. The composition of claim 1 in the form of an aqueous solution.

12. The composition of claim 11 in the form of an aqueous solution comprising from 0.5 to 20 percent of dissolved component a) and from 0.5 to 15 percent of dissolved component b), each percentage being based on the total weight of the aqueous solution.

13. The composition of claim 1 in the form of a gel.

14. A method of reducing or preventing syneresis induced by temperature change of a gel formed from an aqueous solution of an esterified cellulose ether comprising aliphatic monovalent acyl groups and groups of the formula —C(O)—R—COOH, R being a divalent hydrocarbon group, wherein 1) the degree of neutralization of the groups —C(O)—R—COOH is not more than 0.4, II) the total degree of ester substitution is from 0.03 to 0.70, wherein a cellulose ether having a viscosity of from 1.2 to 200 mPa-s, measured as a 2 weight-% aqueous solution at 20° C., is added to the aqueous solution before the gel is formed.

15. A coated dosage form or a polymeric capsule shell wherein the coating or the polymeric capsule shell is made of the composition of any one of claim 1.
